(19) United States
(12) Patent Application Publication
(10) Pub. No.: US 2011/0263975 A1
(43) Pub. Date: Oct. 27, 2011

(54) FLUORESCENT PROBES HAVING A POLYMERIC BACKBONE

(75) Inventor: Helge Tolleshaug, Oslo (NO)

(73) Assignee: GE HEALTHCARE AS, OSLO (NO)

(21) Appl. No.: 13/141,334

(22) PCT Filed: Dec. 22, 2009
(86) PCT No.: PCT/EP2009/067734

§ 371 Date: Jun. 22, 2011

(30) Foreign Application Priority Data
Dec. 22, 2008 (GB) 0823315.7

(51) Int. Cl. A61B 6/00; A61K 49/00; C07K 14/00

(52) U.S. Cl. 600/431; 530/402; 530/409; 530/408; 424/9.6

(57) ABSTRACT

The present invention relates to quenched fluorescent probes which arc activated by biochemical processes. The probes are designed such that intramolecular quenching occurs in the unactivated probe, but that the quencher moieties are cleaved from the probe under defined conditions rendering the probe fluorescent. Also disclosed are optical imaging agents suitable for in vivo imaging comprising the probes, as well as pharmaceutical compositions and kits, as well as in vivo imaging methods.

### FIELD OF THE INVENTION

The present invention relates to quenched fluorescent probes which are activated by biochemical processes. The probes are designed such that intramolecular quenching occurs in the unactivated probe, but that the quencher moieties are cleaved from the probe under defined conditions rendering the probe fluorescent. Also disclosed are optical imaging agents suitable for in vivo imaging comprising the probes, as well as pharmaceutical compositions and kits, as well as in vivo imaging methods.

### BACKGROUND TO THE INVENTION

U.S. Pat. No. 6,083,485 and counterparts discloses in vivo near-infrared (NIR) optical imaging methods using cyanine dyes having an octanol-water partition coefficient of 2.0 or less. Also disclosed arc conjugates of said dyes with “biological detecting units” of molecular weight up to 30 kDa which bind to specific cell populations, or bind selectively to receptors, or accumulate in tissues or tumours. The dyes of U.S. Pat. No. 6,083,485 may also be conjugated to macromolecules, such as polylysine, dextran, polyethylene glycol, methoxypolyethylene glycol, polyvinyl alcohol, dextran, carboxydextran or a cascade polymer-like structure (of molecular weight from 100 Da to over 100 kDa) No specific dye-conjugates are disclosed.

U.S. Pat. No. 6,083,486 (General Hospital Corporation) discloses NIR fluorescent probes that emit substantial fluorescence only after interaction with a target tissue in vivo (i.e. activation). The probes are intramolecularly-quenched, and comprise a polymeric backbone and a plurality of NIR fluorochromes covalently linked to the backbone. The fluorochromes are maintained in a position relative to each other that permits them to interact by energy transfer, and thus quench each other's fluorescence. The probe is designed such that either: (i) the fluorochromes are linked to the backbone by non-biodegradable linkages, but the backbone contains an activation site; or (ii) the fluorochromes are linked to the backbone by linkages which contain an activation site. The “fluorescence activation site” of U.S. Pat. No. 6,083,486 is a covalent bond within the probe which is cleavable by an enzyme present in a target tissue, i.e. is subject to specific enzymatic cleavage. The polymeric backbone is preferably a polypeptide, such as polylysine. Option (ii) is said to be preferred, such that the enzymatic cleavage (activation) liberates fluorochrome molecules from being held in a fluorescence-quenching position. Enzymatic cleavage is thus designed to release the fluorochromes from the probe, so that the fluorescence quenching arrangement is removed, and consequently fluorescence is observed selectively at the enzyme activation sites in vivo.

WO 2004/028449 discloses non-fluorescent bisazulene dimers useful as quenchers of fluorochromes. Also described are fluorescence probes which comprise the bisazulene quenchers and NIR fluorochrome linked to a spacer, wherein the quencher and fluorochrome are separated by a target-specific activation site. Metabolism and bond cleavage at the activation site then causes disruption of the fluorochrome-quencher interaction in target tissue, with consequent fluorescence from the liberated fluorochrome (FIG. 3 of WO 2004/028449).

WO 2007/109364 discloses quenched fluorochrome conjugates and methods of use thereof in the detection and treatment of disorders characterised by unwanted cellular proliferation including cancer. The fluorochrome conjugates comprise a dendrimer and at least two fluorochromes, each covalently linked via a protease cleavage site to the dendrimer at quenching positions. Preferably, at least one of the fluorochromes is a photosensitiser. The dendrimer helps confer the necessary geometry on the conjugated fluorochromes to ensure quenching in the unmodified conjugate. As with U.S. Pat. No. 6,083,486, the metabolisable group may be either within the backbone or in the linkage conjugating the fluorochrome to the dendrimer backbone.

US 2007/0036725 A1 discloses activity based probes for labelling an active protease, which are of formula:

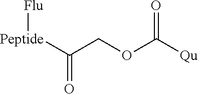

where:
(a) Peptide is a single amino acid, a dipeptide, a tripeptide, or a tetrapeptide and further comprises a capping group;
(b) Qu is either a quencher or a capping group, which capping group is selected from the group consisting of aliphatic ester, aromatic ester or heterocyclic ester;
(c) Flu is a fluorophore, with the proviso that a fluorophore quencher pair may be reversed so that the quencher is on the peptide and the fluorophore is at “Q;” and
(d) any fluorophore and quencher is linked through a lower alkyl, aryl, or aryl-lower alkyl linking group.

WO 2007/008080 describes dual targeting optical imaging contrast agents which comprises:
a target binding ligand (V),
an enzyme cleavable group (E),
a fluorophore (D) and
a quencher agent (Q),
   conjugated with each other in one molecule. The contrast agent preferably comprises the building blocks (i) E-Q and (ii) V-D, conjugated with each other. A most preferred such contrast agent is of formula Q-L¹-E-L²-V-L³-D, where L¹, L² and L³ are independently linker groups.

U.S. Pat. No. 7,329,505 discloses short peptide sequences comprising a peptide backbone having conjugated thereto a fluorochrome and tryptophan (as a quencher). Activation of the probe is via degradation of the peptide backbone by exopeptidase enzymes. That process liberates separate peptide fragments in which the fluorochrome and tryptophan are separated, causing markedly-increased fluorescence. The labelled peptides are said to be useful for the detection of an exopeptidase.

WO 2008/078190 discloses activatable probes which comprise a fluorophore-polymer conjugate, wherein the fluorophores are bound to the polymer via metabolisable linkers, and the polymer further comprise water-solubilising groups. Fluorescence at specific target sites is due to cleavage of the fluorophorc from the polymer. The water-solubilising groups are said to help reduce the intrinsic fluorescence of the native polymer, so that the change in fluorescence on enzymatic cleavage is more pronounced.

### The Present Invention.

The present invention provides quenched fluorescent probes which are activated by biochemical processes. The probes are designed such that intramolecular quenching occurs in the unactivated probe, but that the quencher moieties are cleaved from the probe under defined conditions rendering the probe fluorescent.

Prior art activatable fluorescent probes typically consist of fluorochromes covalently bound to a polymer (e.g., poly-L-lysine) by a short sequence that is enzyme-cleavable (e.g. by cathepsin B). When the fluorochromes are attached in close proximity to one another on the polymer, their fluorescence will be subject to auto-quenching, so that the fluorescence of the polymer construct is low (but usually not zero). Following release of the fluorochromes by enzymatic action, their fluorescence will be much increased. The background fluorescence from the intact probe is a disadvantage. Another drawback is the fact that the liberated fluorochromes tend to diffuse away from the site of activation.

Thus, US 2007/0036725 and WO 2007/008080 both describe:
(1) release of a fluorochrome from quenching;
(2) minimising the movement of the active fluorochrome from the point at which it was de-quenched.
   In US 2007/0036725, the active fluorochrome becomes covalently attached to the active site of an enzyme (para. 0009, p. 1, bottom of col. 1). In WO 2007/008080, the active fluorochrome is attached to a target-binding ligand. In the present invention, movement of the active fluorochrome is impeded by remaining attached to a high-molecular weight polymer (slow diffusion). In contrast, the present invention provides fluorochromes permanently attached to the polymer backbone at intervals such that quenching by fluorescence resonance energy transfer does not occur when only the fluorochromes are attached to the polymer. The fluorescence energy transfer is very sensitive to the distance between the fluorochromes. On exposure to an enzyme, the quenching moieties will be cleaved off and diffuse away from the site, leaving a large and slowly diffusing fluorescent polymer. This will improve the sensitivity and specificity of the probe over probes that generate freely diffusible fluorochromes.

In US 2007/0036725, the scaffolds comprise mono-, di-, tri- or tetrapeptides (para. 0011). In WO 2007/008080, oligomers (of up to about 10 units) may be used as linkers (p. 16). In both of these prior art disclosures, attachment of the optical imaging agent to a polymer would be disadvantageous, because immobilisation of the first of the imaging agents would be likely to impede subsequent reactions by steric hindrance. Attachment of several imaging agents to a high-molecular weight polymer is not mentioned. In the present disclosure, the high molecular weight scaffold:
(1) impedes movement away from the point at which fluorescence is generated;
(2) tends to keep the imaging agent in an area with a high concentration of the enzyme that splits off the quenching moiety.

### DETAILED DESCRIPTION OF THE INVENTION

In a first aspect, the present invention provides an intramolecularly-quenched fluorescence probe comprising a polymeric backbone of molecular weight 10 to 100 kDa and:
(i) a number (z) of near-infrared fluorochromes each covalently linked to the backbone via a first linkage which is resistant to enzyme cleavage and biochemical oxidation;
(ii) a number (z) of quencher moieties each covalently linked to the backbone via a second linkage which is cleavable by either enzyme metabolism or biochemical oxidation;
   wherein z is an integer of value 1 to 150, and wherein said quencher moieties are in a fluorescence-quenching energy transfer relationship with said fluorochromes.

By the term “probe” is meant a compound useful for detecting enzyme activity or biochemical reactive species in vitro or in vivo. A number of enzymes are upregulated in disease, for instance cancers and atherosclerotic diseases. These and other diseases are accompanied by inflammatory conditions, in which oxygen radicals, such as superoxide anion, are evolved by activated immune cells. Accordingly, probes that detect increased amounts of specific enzymes and oxygen radicals are useful for detecting said diseases.

By the term “polymeric backbone” is meant a biocompatible polymer to which the fluorochrome and quencher are linked. The polymer backbone may be a polypeptide, which may comprise different amino acid residues or the same amino acid (i.e. a polyamino acid); a protein; a polysaccharide; a polyester; a polyamidoamine; a polyacrylic acid; a polyalcohol or chitosan. The polymer backbone may comprise D- or L-amino acids. By the term “biocompatible” is meant physiologically tolerable, i.e. can be administered to the mammalian body without toxicity or undue discomfort. The molecular weight of the polymeric backbone is suitably 10 to 100 kDa (10,000 to 100,000 Da). The molecular weight of the probe is high in order to retard diffusion, but not so high as to prevent it from transport into tissues or tumours from the circulation.

The term “fluorochrome” has its conventional meaning, i.e. a fluorescent dye. Suitable fluorochromes for use in the present invention are fluorescent dyes having an absorbance maximum in the range 600-1000 nm and emission maxima in the range 600-1200 nm.

By the term “fluorescence quencher” is meant a moiety which suppresses the fluorescence of the fluorochrome such that the unactivated probe having both quenchers and fluorochromes attached would have minimal fluorescence. Quencher molecules are known in the art [Johansson, Meth. Mol. Biol., 335, 17-29 (2006), and Bullok et al Biochem., 46(13), 4055-4065 (2007)]. Pairs of fluorochromes that are susceptible to fluorescence resonance energy transfer are described by Shanker et al [Meth. Cell Biol., 84, Chapter 8, 213-242 (2008)] and in Lakowicz (“Principles of Fluorescence Spectroscopy” 2^{nd} edition Kluwer, (1999), p. 388]. Shanker and Lakowicz also include procedures for finding new fluorochrome/quencher pairs.

By the term “intramolecularly-quenched” is meant that within the intact probe molecule the fluorochromes and quencher moieties are arranged such that any fluorescence from the fluorochromes is quenched.

By the term “biochemical oxidation” is meant an oxidation process generated by to cells or organs of the mammalian body in vivo or in vitro. Suitable oxidation processes of this nature include oxidation by extracellular superoxide anion, hydrogen peroxide or hydroxyl radicals which are capable of cleaving disulfide bonds and also carbon-carbon double bonds in certain configurations such as those that occur in polyunsaturated fatty acids.

By the term “resistant to enzyme cleavage or biochemical oxidation” is meant a covalent bond which is not a substrate for enzymes of the mammalian body, and is not cleaved by the biochemical oxidation processes described above. Examples of suitable such bonds for the first linkage are carbon-carbon bonds; ether bonds; thioether bonds; sulfonamide bonds and amide bonds (excluding peptide bonds). Clearly, the first linkage excludes those cleavable bonds which fall within the definition of the second linkage (see below).

By the term “cleavable by either enzyme metabolism or biochemical oxidation” is meant that the second linkage can be cleaved by either: (a) an enzyme of the mammalian body in vivo or in vitro; or (b) the biochemical oxidation processes defined above. The enzymatic reaction of (a) may be cleavage of a natural substrate of the enzyme or an analogue of the substrate. The enzymes may include: esterases, endopeptidases, endoproteinases, dealkylases, glycosidases, endoglycanases, heparinases, chondroitinases, hyaluronidases, RNAses, DNAses or phosphodiesterases.

By the term “fluorescence-quenching energy transfer relationship” is meant that the fluorochrome and quencher are maintained in a position relative to each other that permits them to interact by energy transfer to permit quenching to occur.

By the term “quencher moiety” is meant a moiety which suppresses the fluorescence of the fluorochrome such that the intact probe has minimal fluorescence. Thus, upon irradiation with light of a wavelength which excites the fluorochrome, the quencher absorbs the energy of the excited fluorochrome such that overall the intact probe has minimal fluorescence. Quenchers suitable for use in the present invention include:
(a) non-fluorescent dyes such as DABCYL [4-(4′-dimethylaminobenzencazo)benzoic acid; which absorbs in the region 360 to 560 nm] and other azo dyes; DANSYL, QSY-7, Black Hole Quenchers, etc.,
(b) fluorophores with suitable absorption spectra;
(c) nitro-substituted phenyl moieties including p-nitrobenzoic acid, m-nitrobenzoic acid, o-nitrobenzoic acid, 3,5-dinitrobenzoic acid, and 2,4,6-trinitrobenzenesulfonic acid;
(d) azulene dimers.
   Quencher molecules are known in the art [Johansson, Meth. Mol. Biol., 335, 17-29 (2006); Marras ibid, 335, 3-16 (2006), and Bullok et al (above)].

The probes of the present invention are designed such that pairs of fluorochromes in the cleaved probe, i.e. after removal of the quencher moiety(ies) are not in a self-quenching relationship. Note that, if some self-quenching of fluorochromes occurs in the intact probe, that is not a problem, since minimal fluorescence is desirable for the intact probe anyway. The activation of the probes is shown schematically in FIG. 1:

FIG. 1: activation of probes

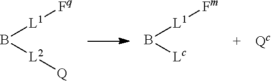

where:
B is the polymeric backbone;
L¹ is the first linkage;
L² is the second linkage;
L^{c} is the residue of the cleaved second linkage;
Q is the quencher moiety;
Q^{c} is the cleaved quencher moiety, including any residue of the cleaved second linkage;
F^{q} is the fluorochrome in a quenched relationship with Q;
F^{m} is the fluorochrome in an environment where it can fluoresce.

The number of pairs of fluorochrome and quencher moiety per probe is suitably in the range 1 to 150. Preferably, the probe has at least 2 or 3 such pairs. For the upper limit (150), the probe would have a molecular weight of approximately 100 kDa.

The probes of the first aspect are preferably used in optical imaging agents suitable for in vivo imaging, as is described in the second aspect (below). The probes may, however, also have in vitro applications (eg. assays quantifying the cleavage enzyme in biological samples or visualisation of such enzymes in tissue samples).

The probes of the invention may optionally further comprise a biological targeting moiety. The biological targeting moieties might be attached to the backbone, to L¹ of FIG. 1, or to the fluorochrome. By the term “biological targeting moiety” (BTM) is meant a compound which, after in vivo administration, is taken up selectively or localises at a particular site of the mammalian body. Such sites may for example be implicated in a particular disease state be indicative of how an organ or metabolic process is functioning. The biological targeting moiety preferably comprises: 3-100 mer peptides, peptide analogues, peptoids or peptide mimetics which may be linear peptides or cyclic peptides or combinations thereof; or enzyme substrates, enzyme antagonists or enzyme inhibitors; synthetic receptor-binding compounds; oligonucleotides, or oligo-DNA or oligo-RNA fragments.

By the term “peptide” is meant a compound comprising two or more amino acids, as defined below, linked by a peptide bond—i.e. an amide bond linking the amine function of one amino acid to the carboxyl of another amino acid, as well as the carboxyl being linked to C-2 (or “alpha carbon”) of the respective amino acids. The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. The term “peptide analogue” refers to peptides comprising one or more amino acid analogues, as described below. See also “Synthesis of Peptides and Peptidomimetics”, M. Goodman et al, Houben-Weyl E22c, Thieme.

By the term “amino acid” is meant an L- or D-amino acid, amino acid analogue (eg. naphthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)].

Suitable enzyme substrates, antagonists or inhibitors include glucose and glucose analogues such as fluorodeoxyglucose; fatty acids, or elastase, Angiotensin II or metalloproteinase inhibitors. A preferred non-peptide Angiotensin II antagonist is enalapril. Losartan is a non-peptidic antagonist of the angiotensin II receptor. Suitable synthetic receptor-binding compounds include estradiol, estrogen, progestin, progesterone and other steroid hormones; ligands for the dopamine D-1 or D-2 receptor, or dopamine transporter such as tropanes; and ligands for the serotonin receptor.

### Preferred Features.

The molecular weight of the polymeric backbone is preferably 9 to 50 kDa, more preferably 10 to 40 kDa, most preferably 11 to 35 kDa. The probe of the present invention preferably has a molecular weight of about 30 to 50 kDa, which favours slow elimination via the kidneys. The probe is preferably suitable for in vivo applications.

The probe of the present invention is preferably hydrophilic in order to increase solubility in water and decrease aggregation. The probe should also preferably have a net negative charge in order to decrease non-specific attachment to cell surfaces (which are always negatively charged) and facilitate clearance by the kidneys in vivo.

The quencher moiety is preferably a different chemical species to the fluorochrome. The quencher moiety is most preferably non-fluorescent, i.e. does not fluoresce when irradiated with light of the wavelength suitable for activating the fluorochrome, neither does it fluoresce when irradiated with light that is emitted from the fluorochrome. In that way, once the quencher is cleaved from the probe it does not provide any competing fluorescence—any fluorescence is entirely due to the activated probe. For in vivo applications, the quencher is suitably biocompatible (as defined above), and hence the liberated quencher is non-toxic to the mammalian body. Hence, preferred quencher moieties for in vivo applications are both non-fluorescent and biocompatible. Preferred non-fluorescent quenchers include the azulene dimers taught by Weissleder et al [Ang. Chem. Int. Ed. Eng., 41, 3659-3662 (2002) and WO 04/028449]. The far-red quencher QSY 21 is taught by Bullok et al [Biochem., 46(13), 4055-4065 (2007)]. As fluorochromes that emit light in the near-infrared range are especially suitable for use in vivo, quenchers that absorb in this range are preferred. Quenchers should be rapidly eliminated by the kidneys. Elimination might follow a conjugation reaction such as glucuronidation in the liver.

The probe is preferably chosen such that the second linkage is cleavable by the action of enzyme(s). Preferred such enzymes are hydrolytic enzymes, more preferably a peptidase or protease. Most preferred cleavage enzymes for use in the present invention include: cathepsin B, D, K, L and S; matrix metalloproteinases; urokinase-type plasminogen activator; kallikreins; hepsin; furin; matriptase; procollagen convertases; bone morphogenetic protein-1, and tolloid-like proteinases 1 and 2. Many of these enzymes are involved in invasion by tumours. Specific enzyme substrate linkages are given in Tables 1 and 2:

TABLE 1

| Enzyme target | Substrate Sequence |
|---|---|
| MMP-1 | Pro-Leu-Gly/Leu-Trp-Ala-D-Arg-NH₂ |
| MMP-2 | Lys-Pro-Leu-Ala/Nva-Asp-Ala-Arg-NH₂ or |
| | Pro-Leu-Gly/Leu-Ala-Arg-NH₂. |
| MMP-3 | Arg-Pro-Lys-Pro-Tyr-Ala/Nva-Trp-Met-Lys-NH₂ or |
| | Arg-Pro-Lys-Pro-Val-Glu/Nva-Trp-Arg-Lys-NH₂. |
| MMP-7 | Arg-Pro-Leu-Ala/Leu-Trp-Arg-Ser(AHX)-Cys or |
| | Arg-Pro-Leu-Ala/Leu-Trp-Arg-Ser or |
| | Pro-Leu-Gly-Leu-Dpa-Ala-Arg-NH₂. |
| MMP-9 | Ser-Gly-Lys-Gly-Pro-Arg-Gln/Ile-Thr-Ala or |
| | Ser-Gly-Lys-Ile-Pro-Arg-Arg/Leu-Thr-Ala or |
| | Pro-Gln-Gly-Ile-Ala. |
| MMP-13 | Pro-Cha-Gly/Nva-His-Ala-NH₂ or Pro-Leu-Gly/Leu-Ala-Arg-NH₂ |
| | or Gly-Pro-Leu-Gly-Met-Arg-Gly-Leu-NH₂ |
| uPA | PGSGR/SAG or PGSGR/SASGTTGTG or SGR/SA or GSGK/S |
| Thrombin | D-Phe-Pip-Arg-pNA or D-Phe-Pro-Arg-pNA or |
| | Ac-Nle-Thr-Pro-Arg-AMC or Ac-Val-Thr-Pro-Arg-AMC |
| Cathepsin B | Ala-Arg-Arg-Ala |
| Cathepsin E | Gly-Ser-Pro-Ala-Phe-Leu-Ala-Lys-D-Arg |
| Cathepsin D and E | Gly-Lys-Pro-Ile-Leu-Phe-Phe-Arg-Leu-Lys-D-Arg |
| MMPs, Cathepsin | Lys-Pro-Leu-Gly-Leu-Dap-Ala-Arg |
| D & E, ADAM10 | |
| MMPs, TACE | Lys-Pro-Leu-Gly-Leu-A2pr-Ala-Arg |
| MMPs | Pro-Cha-Gly-Nva-His-Ala-Dap |
| Gamma-secretase | Gly-Gly-Val-Val-Ile-Ala-Thr-Val-Lys-D-Arg-D-Arg-D-Arg |
| Caspase-1 | Tyr-Val-Ala-Asp-Ala-Pro-Lys |
| Caspase-3 | Asp-Glu-Val-Asp-Ala-Pro-Lys |
| Caspases (general) | Asp-Glu-Val-Asp |
| Proteinase A/pepsin | Ala-Pro-Ala-Lys-Phe-Phe-Arg-Leu-Lys |
| Collagenase | Pro-Cha-Gly-Cys(Me)-His-Ala-Lys |
| ADAM17/TACE | Leu-Ala-Gln-Ala-Val-Arg-Ser-Ser-Ser-Arg-Dap |
| ACE2 | Gly-Phe-Ser-Pro-Tyr(NO₂) |

where:
Nva = L-norvaline; AHX = aminohexanoic acid; Dpa = N-3-(2,4-dinidopheny1)-L-2,3-diaminopropionyl quencher moiety; pNA = p-nitroanthne and AMC = aminomethylcoumarin.

Protease-sensitive sequences are given in Table 2:

TABLE 2

| enzyme | sequence | reference |
|---|---|---|
| gelatinase B | A-P^{OH}GPQG#FQGNP^{OH}G | van den Steen et al |
| human tissue kallikrein 1 | Abz-AIKFFSRQ- | Pimenta et al |
| prostase/KLK4 | R-Q\L\V-Q\S\V-I\V | Matsumura et al |
| cathepsin B | XRRX, XKX | |
| kallikreins | PFRX or D-VLRX | |
| cathepsin D | RGFFL or RGFFP | |
| neutral endopeptidase | AAF or AGLA | |
| MMP-2 | PKPQQFFGLK(Dnp)G | |
| MMP-3 | RPKPVD-Nva-WRK(Dnp) | |
| MMP-9 | P^{OH}GPDGFQGNP^{OH}G | |
| prostate-specific antigen | LRLSSYYSG | |
| cathepsin L | FRRX | |
| human kallikrein 5 | GPRX | |
| furin and similar | RRPR or RRKR | Khatib et al |
| enzymes | | |
| cathepsins B, L | ZFRmca, abz-FR-Etdiam-Dnp | Melo et al |
| prostate-specific antigen | GISSFY#SSTEERLW | Coombs et al |
| hepsin | ZFRmca, abz-FR-Etdiam-Dnp | |
| ADAMTS−1,−4,−5,−8,−9,−15 | PLPRNXTEXE#ARGXVILTXK | Jones & Riley, |
| | | Sandy et al. |

# = cleavage point, X = any amino acid, P^{OH} = hydroxyproline, Dnp = dinitrophenyl

Coombs et al Substrate specificity of prostate-specific antigen (PSA). Chemistry & Biology 5, no. 9:475-488 (1998).

Khatib et al, Proprotein Convertases in Tumor Progression and Malignancy: Novel Targets in Cancer Therapy. Am. J. Patho. 160, no. 6:1921-1935 (2002).

Matsumura et al. Substrates of the prostate-specific serine protease prostase/KLK4 defined by positional-scanning peptide libraries. The Prostate 62, no. 1.1-13 (2005).

Melo et al Synthesis and hydrolysis by cysteine and serine proteases of short internally quenched fluorogenic peptides. Analytical Biochemistry 293, no. 1:71-77 (2001).

Other peptidases and proteinases that could be used in the present context include plasmin, urokinase plasminogen activator, prostatin, testisin, TSP50, GPI-SP1-3, TESP 1-2, DISP, tryptase γ1 matriptase-1, 2 and 3, hepsin, TMPRSS 2, 3 and 4, spinesin, HAT, MSPL, kallikrein-14, polyserase-1, furin, PACE4, PCI, PC5, PC7, kallikrein-5, caspase 9, cathepsins H, K and L, and DESC-1.

The backbone of the probe is preferably resistant to enzyme cleavage, i.e. it is stable in vivo. That may be achieved eg. by the use of polymers of unnatural amino acids such as 2,3-diaminopropionic acid or 2,4-diaminobutyric acid. Alternatively, one or both termini of the peptide, preferably both, have conjugated thereto a metabolism inhibiting group (M^{IG}). By the term “metabolism inhibiting group” (M^{IG}) is meant a biocompatible group which inhibits or suppresses enzyme activity, especially activity of peptidases such as carboxypeptidases, which otherwise might cleave amino acids from the peptide at either the amino terminus or carboxy terminus. Such groups are particularly important for in vivo applications, and are known to those skilled in the art and are suitably chosen from, for the peptide amine terminus:

N-acylated groups —NH(C═O)R^{G} where the acyl group —(C═O)R^{G} has R^{G} chosen from: C₁₋₆ alkyl, C₃₋₁₀ aryl groups or comprises a polyethyleneglycol (PEG) building block. Suitable PEG groups are described below. Preferred such PEG groups are the biomodifiers of Formulae Biol or Bio2 (below). Preferred such amino terminus M^{IG} groups are acetyl, benzyloxycarbonyl or trifluoroacetyl, most preferably acetyl.

Suitable metabolism inhibiting groups for the peptide carboxyl terminus include: carboxamide, tert-butyl ester, benzyl ester, cyclohexyl ester, amino alcohol or a polyethyleneglycol (PEG) building block. A suitable M^{IG} group for the carboxy terminal amino acid residue of the BTM peptide is where the terminal amine of the amino acid residue is N-alkylated with a C₁₋₄ alkyl group, preferably a methyl group. Preferred such M^{IG} groups are carboxamide or PEG, most preferred such groups are carboxamide.

Preferred PEG groups comprise units derived from oligomerisation of the monodisperse PEG-like structures of Formulae Bio1 or Bio2:

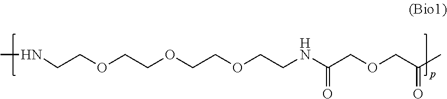

17-amino-5-oxo-6-aza-3, 9, 12, 15-tetraoxaheptadecanoic acid of Formula Bio1

wherein p is an integer from 1 to 10. Alternatively, a PEG-like structure based on a propionic acid derivative of Formula Bio2 can be used

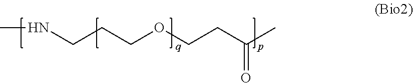

where p is as defined for Formula Bio1 and q is an integer from 3 to 15.

In Formula Bio2, p is preferably 1 or 2, and q is preferably 5 to 12.

Such resistance of the probe backbone to cleavage has the advantage that, once activated, the relatively high molecular weight fluorochrome-containing portion does not diffuse away readily, whereas the relatively low molecular weight quencher-containing fragment diffuses away more rapidly. Thus, the probe will tend to remain in proximity to the target of interest, thus giving target-selective imaging. The backbone preferably comprises a polypeptide and/or a copolymer. The copolymer has the advantage that the two components of the copolymer can be chosen to have different functional groups which permit the chemical distinction between the first and second linkages. A preferred such copolymer comprises a lysine-glutamic acid copolymer. In that way, the fluorophore can be attached to the lysine residues (eg. via a sulfonamide or carboxamide bond), while a short cleavable sequence attached to the glutamic acid residues terminates in the quenching moiety. The performance might be improved by including “spacers”, such as additional amino acids or ethylene glycol oligomers, in order to increase the distance between the fluorophores following enzymatic action:

[Lys(L¹-F⁴)-Glu(L²-Q)-Ala-Ala]ₙ→[Lys(L¹-F^{m})-Glu(L^{c})-Ala-Ala]ₙ+Q^{c}

where:

F^{q} is as defined for FIG. 1, and is attached via L¹ at the 6-position of lysine;

L¹, L², L^{c}, F^{m}, Q and Q^{c} are as defined for FIG. 1;

n is an integer of value 2 to 150.

In the cleaved product on the right, the fluorochromes (F^{m}) are separated by three amino acids and accordingly have minimal susceptibility to self-quenching. The efficiency of resonance energy transfer decreases as the sixth power of the distance [Shanker et al, Meth. Cell Biol., 84, Chapter 8, 213-242 (2008)].

The probe may optionally be further improved by choosing substituents so that the substrate polymer is negative charged (and freely soluble), and the cleaved product probe is positively charged, thus conferring a tendency for the cleaved probe to adhere to cell surfaces. This might be achieved by choosing releasable quenchers that are heavily sulfonated or carboxylated.

Preferred fluorochromes have an extensive delocalized electron system, eg. cyanine dyes, merocyanine dyes, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include fluorescein, sulforhodamine 101 (Texas Red), rhodamine B. rhodamine 6G, rhodamine 19, indocyanine green, Cy2, Cy3B, Cy3.5, Cy5, Cy5.5, Cy7, Cy7.5, Marina Blue, Pacific Blue, Oregon Green 488, Oregon Green 514, tetramethylrhodamine, and Alexa. Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. The cyanine dyes are particularly preferred. Licha et al have reviewed dyes and dye conjugates for in vivo optical imaging [Topics Curr. Chem., 222, 1-29 (2002); Adv. Drug Deliv. Rev., 57, 1087-1108 (2005)].

Preferred cyanine dyes which are fluorophores are of Formula II:

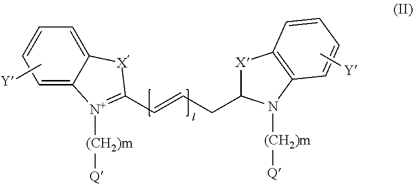

wherein:

each X′ is independently selected from: —C(CH₃)₂, —S—, —O—or
—C[(CH₂)₄CH₃][(CH₂)_{b}M]—, wherein a is an integer of value 0 to 5, b is an integer of value 1 to 5, and M is group G or is selected from SO₃M¹ or II;

each Y′ independently represents 1 to 4 groups selected from the group consisting of:
H, —CH₂NH₂, —SO₃M¹, —CH₂COOM¹, —NCS and F, and wherein the Y′ groups are placed in any of the positions of the aromatic ring;

Q′ is independently selected from the group consisting of: H, SO₃M¹, NH₂, COOM¹,
ammonnium, ester groups, benzyl and a group G;

M¹ is H or B^{c}, where B^{c} is a biocompatible cation;

1 is an integer from 1 to 3;

and m is an integer from 1 to 5;

wherein at least one of X′, Y′ and Q′ comprises a group G;

G is a reactive or functional group suitable for attaching to the backbone.

By the term “biocompatible cation” (B^{c}) is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

When a biological targeting molecule (BTM) is attached, the BTM may be of synthetic or natural origin, but is preferably synthetic. The term “synthetic” has its conventional meaning, ie. man-made as opposed to being isolated from natural sources eg. from the mammalian body. Such compounds have the advantage that their manufacture and impurity profile can be fully controlled. Monoclonal antibodies and fragments thereof of natural origin are therefore outside the scope of the term ‘synthetic’ as used herein. When the BTM is a peptide, preferred such peptides include:
somatostatin, octreotide and analogues,
peptides which bind to the ST receptor, where ST refers to the heat-stable toxin produced by E. coli and other micro-organisms;
laminin fragments eg. YIGSR, PDSGR, IKVAV, LRE and KCQAGTFALRGDPQG,
N-formyl peptides for targeting sites of leucocyte accumulation,
Platelet factor 4 (PF4) and fragments thereof,
RGD (Arg-Gly-Asp)-containing peptides, which may eg. target angiogenesis [R. Pasqualini et al., Nat. Biotechnol. 1997 June;15(6):542-6]; [E. Ruoslahti, Kidney Int. 1997 May;51(5):1413-7].
peptide fragments of α₂-antiplasmin, fibronectin or beta-casein, fibrinogen or thrombospondin. The amino acid sequences of α2-antiplasmin, fibronectin, beta-casein, fibrinogen and thrombospondin can be found in the following references: α₂-antiplasmin precursor [M. Tone et al., J. Biochem, 102 1033, (1987)]; beta-casein [L. Hansson et al, Gene, 139, 193, (1994)]; fibronectin [A. Gutman et al, FEBS Lett., 207, 145, (1996)]; thrombospondin-1 precursor [V. Dixit et al, Proc. Natl. Acad. Sci., USA, 83, 5449, (1986)]; R. F. Doolittle, Ann. Rev. Biochem., 53, 195, (1984);
peptides which are substrates or inhibitors of angiotensin, such as: angiotensin II Asp-Arg-Val-Tyr-Ile-His-Pro-Phe (E. C. Jorgensen et al, J Med. Chem., 1979, Vol 22, 9, 1038-1044)
[Sar, Ile] Angiotensin II: Sar-Arg-Val-Tyr-Ile-His-Pro-Ile (R. K. Turker et al., Science, 1972, 177, 1203).
Angiotensin I: Asp-Arg-Val-Tyr-Ile-His-Pro-Phe-His-Leu;
endothelins, e.g. endothelin-1. Cys-Ser-Cys-Ser-Ser-Leu-Met-Asp- Lys-Glu-Cys-Val-Tyr-Phe-Cys-His-Leu-Asp-Ile-Ile-Trp
bombesin: Xaa-Gln-Arg-Leu-Gly-Asn-Gln-Trp-Ala-Val-Gly-His-Leu-Met-NH₂
gastrin: pyroglutamyl-Gly-Pro-Trp-Leu-Glu-Glu-Glu-Glu-Glu-Ala-Tyr-Gly-Trp-Met-Asp-Phe-NH₂
gonadotropin-releasing hormone: pyroglutamyl-His-Trp-Ser-Tyr-Gly-Leu-Arg-Pro-Gly-NH₂

The use of a BTM as a part of the probe has the advantage of further increasing the specificity of the probe.

When the BTM is a peptide, one or both termini of the peptide, preferably both, have conjugated thereto a metabolism inhibiting group (M^{IG})—as defined above.

Peptides of the invention which are not commercially available can be synthesised by solid phase peptide synthesis as described in P Lloyd-Williams, F. Albericio and E. Girald; Chemical Approaches to the Synthesis of Peptides and Proteins, CRC Press, 1997.

The probes of the invention can be prepared as follows: Poly-L-lysine, dextrans, amylase (poly-D-glucose), chitosan, RNA DNA, polylactic acid, poly(lactic/glycolic) acid, heparan sulphate and chondroitin sulphate are commercially available. Chitosan, heparan sulphate and chondroitin sulphate might be de-acetylated or de-sulfated in order to make amino groups available for reaction and to increase the number of reactive groups; such methods are well known in the art. Polymers of amino acids with an amino group in the side chain would need to be synthesised. The outline of a suitable method is a follows: a blocking group is introduced on the amino group of the side chain, and the substituted amino acid is polymerised in aqueous solution at slightly acid pH, using a water-soluble carbodiimide. The blocking group is removed, and by-products are removed on a size exclusion column, coincidentally narrowing the range of molecular weights in the product.

In order to facilitate conjugation to the backbone, the fluorochrome and/or the quencher moiety each suitably has attached thereto a reactive functional group (Q^{a}). The Q^{a} group is designed to react with a complementary functional group of the backbone, thus forming a covalent linkage. The complementary functional group of the backbone may he an intrinsic part of the backbone, or may be introduced by use of derivatisation with a bifunctional group as is known in the art. Table 3 shows examples of reactive groups and their complementary counterparts:

TABLE 3
Reactive Substituents and Complementary
Groups Reactive Therewith.

| Reactive Group (Q^{a}) | Complementary Group(s) |
|---|---|
| Activated ester | primary amino, secondary amino |
| acid anhydride, acid halide. | primary amino, secondary amino, hydroxyl |
| isothiocyanate | amino groups |
| vinylsulphone | amino groups |
| dichlorotriazine | amino groups |
| haloacetamide, malcimide | thiol, imidazole, hydroxyl, amines, |
| | thiophosphate |
| carbodiimide | carboxylic acids |
| hydrazine, hydrazide | carbonyl including aldehyde and ketone |
| phosphoramidite | hydroxyl groups |

By the term “activated ester” or “active ester” is meant an ester derivative of the carboxylic acid which is designed to be a better leaving group, and hence permit more facile reaction with nucleophiles, such as amines. Examples of suitable active esters are: N-hydroxysuccinimide (NHS), pentafluorophenol, pentafluorothiophenol, para-nitrophenol and hydroxybenzotriazole. Preferred active esters are N-hydroxysuccinimide or pentafluorophenol esters.

Examples of functional groups present in the backbone include: hydroxy, amino, sulphydryl, carbonyl (including aldehyde and ketone) and thiophosphate. Suitable Q^{a} groups may be selected from: carboxyl; activated esters; isothiocyanate; maleimide; haloacetamide; hydrazide; vinylsulphone, dichlorotriazine and phosphoramidite. Preferably, Q^{a} is an activated ester of a carboxylic acid, an isothiocyanate, a maleimide or a haloacetamide.

When the complementary group is an amine or hydroxyl, Q^{a} is preferably an activated ester, with preferred such esters as described above. When the complementary group is a thiol, Q^{a} is preferably a maleimide or iodoacetamide group.

General methods for conjugation of fluorochromes and optical dyes to biological molecules are described by Licha et at [Topics Curr. Chem., 222, 1-29 (2002); Adv. Drug Deliv. Rev., 57, 1087-1108 (2005)]. Peptide, protein and oligonucleotide substrates for use in the invention may be labelled at a terminal position, or alternatively at one or more internal positions. For reviews and examples of protein labelling using fluorescent dye labelling reagents, see “Non-Radioactive Labelling, a Practical Introduction”, Garman, A. J. Academic Press,1997; “Bioconjugation—Protein Coupling Techniques for the Biomedical Sciences”, Aslam, M. and Dent, A., Macmillan Reference Ltd, (1998). Protocols are available to obtain site specific labelling in a synthesised peptide, for example, see Hermanson, G. T., “Bioconjugate Techniques”, Academic Press (1996).

In the conjugation reaction, the backbone, fluorochrome or quencher may optionally have any additional functional groups which could potentially react protected with suitable protecting groups so that chemical reaction occurs selectively at the desired site only. By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Amine protecting groups are well known to those skilled in the art and are suitably chosen from: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl). Suitable thiol protecting groups are Trt (Trityl), Acm (acetamidomethyl), t-Bu (tert-butyl), tert-Butylthio, methoxybenzyl, methylbenzyl or Npys (3-nitro-2-pyridine sulfenyl). The use of further protecting groups are described in ‘Protective Groups in Organic Synthesis’, Theodora W. Greene and Peter G. M. Wuts, (John Wiley & Sons, 1991). Preferred amine protecting groups are Boc and Fmoc, most preferably Boc. Preferred amine protecting groups are Trt and Acm.

Methods of conjugating optical reporter dyes, to amino acids and peptides are described by Licha (vide supra), as well as Flanagan et al [Bioconj.Chem., 8, 751-756 (1997)], Lin et al, [ibid, 13, 605-610 (2002)] and Zaheer [Mol. Imaging, 1(4), 354-364 (2002)].

In a second aspect, the present invention provides an optical imaging agent suitable for in vivo imaging which comprises the probe of the first aspect.

By the term “imaging agent” is meant a compound suitable for optical imaging of a region of interest of the whole (ie. intact) mammalian body in vivo. Preferably, the mammal is a human subject. The imaging may be invasive (eg. intra-operative or endoscopic) or non-invasive. The imaging may optionally be used to facilitate biopsy (eg. via a biopsy channel in an endoscope instrument), or tumour resection (eg. during intra-operative procedures via tumour margin identification).

Preferably, the imaging agent is provided as a pharmaceutical composition which comprises the imaging agent together with a biocompatible carrier, in a form suitable for mammalian administration. The “biocompatible carrier” is a fluid, especially a liquid, in which the imaging agent can be suspended or dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). Preferably the biocompatible carrier is pyrogen-free water for injection or isotonic saline.

The imaging agents and biocompatible carrier are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour.

Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

The pharmaceutical composition may optionally contain additional excipients such as an antimicrobial preservative, pH-adjusting agent, filler, stabiliser or osmolality adjusting agent. By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The pharmaceutical compositions of the second aspect may be prepared under aseptic manufacture (i.e. clean room) conditions to give the desired sterile, non-pyrogenic product. It is preferred that the key components, especially the associated reagents plus those parts of the apparatus which come into contact with the imaging agent (eg. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition.

The pharmaceutical composition of the second aspect is preferably prepared from a kit, as described for the third aspect below.

In a third aspect, the present invention provides a kit for the preparation of the imaging agent pharmaceutical composition as described in the second aspect. The kit comprises the imaging agent of the first aspect in sterile, solid form such that upon reconstitution with a sterile supply of the biocompatible carrier, dissolution occurs to give the desired pharmaceutical composition.

In that instance, the imaging agent, plus other optional excipients as described above, may be provided as a lyophilised powder in a suitable vial or container. The agent is then designed to he reconstituted with the desired biocompatible carrier to give the pharmaceutical composition in a sterile, apyrogenic form which is ready for mammalian administration.

A preferred sterile, solid form of the imaging agent is a lyophilised solid. The sterile, solid form is preferably supplied in a pharmaceutical grade container, as described for the pharmaceutical composition (above). When the kit is lyophilised, the formulation may optionally comprise a cryoprotectant chosen from a saccharide, preferably mannitol, maltose or tricine.

In a fourth aspect, the present invention provides an in vivo optical imaging method comprising:
(i) administering to a living animal or human subject the optical imaging agent of the second aspect;
(ii) allowing time for (a) the probe to accumulate in a target tissue of interest within said subject, and (b) enzymes in said target tissue to activate the probe by enzymatic cleavage at one or more of the second linkages to give the activated probe;
(iii) illuminating the target tissue with a near-infrared excitation light of a wavelength absorbable by the fluorochrome of said activated probe;
(iv) fluorescence from the activated probe, which is generated by excitation of the fluorochrome in step (iii) is detected using a fluorescence detector;
(v) the light detected by the fluorescence detector is optionally filtered to separate out the fluorescence component; and
(vi) forming an optical image of the target tissue from the detected fluorescence of steps (iv) or (v).

By the term “optical imaging” is meant any method that forms an image for detection, staging or diagnosis of disease, follow up of disease development or for follow up of disease treatment based on interaction with light in the green to near-infrared region (wavelength 500-1200 nm). Optical imaging, further includes all methods from direct visualization without use of any device and involving use of devices such as various scopes, catheters and optical imaging equipment, eg. computer-assisted hardware for tomographic presentations. The modalities and measurement techniques include, but are not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarization, luminescence, fluorescence lifetime, quantum yield, and quenching. Further details of these techniques are provided by: (Tuan Vo-Dinh (editor): “Biomedical Photonics Handbook” (2003), CRC Press LCC; Mycek & Pogue (editors): “Handbook of Biomedical Fluorescence” (2003), Marcel Dekker, Inc.; Splinter & Hopper: “An Introduction to Biomedical Optics” (2007), CRC Press LCC.

The wavelength for excitation using the illumination light of step (iii) varies depending on the particular fluorochrome used, but is typically in the range 500-1200 nm for probes of the present invention, preferably of wavelength 600-1000 nm. The apparatus for generating the excitation light may be a conventional excitation light source such as: a laser (e.g., ion laser, dye laser or semiconductor laser); halogen light source or xenon light source. Various optical filters may optionally be used to obtain the optimal excitation wavelength. Preferably, the excitation light of step (iii) of the fourth aspect is continuous wave (CW) in nature.

The optical imaging method is preferably fluorescence endoscopy. The mammalian body of the fourth aspect is preferably the human body. Preferred embodiments of the imaging agent are as described for the second aspect (above).

A preferred optical imaging method of the fourth aspect is Fluorescence Reflectance Imaging (FRI). In FRI, the imaging agent of the present invention is administered to a subject to be diagnosed, and subsequently a tissue surface of the subject is illuminated with an excitation light—usually continuous wave (CW) excitation. The light excites the fluorochrome of the imaging agent. Fluorescence from the imaging agent, which is generated by the excitation light, is detected using a fluorescence detector. The returning light is preferably filtered to separate out the fluorescence component (solely or partially). An image is formed from the fluorescent light. Usually minimal processing is performed (no processor to compute optical parameters such as lifetime, quantum yield etc.) and the image maps the fluorescence intensity. The imaging agent is designed to concentrate in the disease area, producing higher fluorescence intensity. Thus the disease area produces positive contrast in a fluorescence intensity image. The image is preferably obtained using a CCD camera or chip, such that real-time imaging is possible.

In the method of the fourth aspect, the imaging agent or pharmaceutical composition has preferably been previously administered to said mammalian body. By “previously administered” is meant that the step involving the clinician, wherein the imaging agent is given to the patient eg. as an intravenous injection, has already been carried out prior to imaging. This embodiment includes the use of the imaging agent of the second embodiment for the manufacture of a diagnostic agent for the diagnostic imaging in vivo of disease states of the mammalian body.

In a fifth aspect, the present invention provides an in vivo optical imaging method comprising:
(i) administering to a living animal or human subject the optical imaging agent of the second aspect;
(ii) allowing time for (a) the probe to accumulate in a target tissue of interest within said subject, and (b) enzymes in said target tissue to activate the probe by enzymatic cleavage at one or more of the second linkages to give the activated probe;
(iii) exposing light-scattering biologic tissue of said subject having a heterogeneous composition to excitation light from a light source with a pre-determined time varying intensity to excite the imaging agent, the tissue multiply-scattering the excitation light;
(iv) detecting a multiply-scattered light emission from the tissue in response to said exposing;
(v) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(vi) generating an image of the tissue by mapping the heterogeneous composition of the tissue in accordance with the values of step (v).

The imaging method of the fifth aspect uses FDPM (frequency-domain photon migration). This has advantages over continuous-wave (CW) methods in circumstances where greater depth of detection of the dye within tissue is important [Sevick-Muraca et al, Curr. Opin. Chem. Biol., 6, 642-650 (2002)]. For such frequency/time domain imaging, it is advantageous if the fluorochrome has fluorescent properties which can be modulated depending on the tissue depth of the lesion to be imaged, and the type of instrumentation employed.

The fluorescence characteristic of step (v) preferably corresponds to uptake of the imaging agent and preferably further comprises mapping a number of quantities corresponding to adsorption and scattering coefficients of the tissue before administration of the imaging agent. The fluorescence characteristic of step (v) preferably corresponds to at least one of fluorescence lifetime, fluorescence quantum efficiency, fluorescence yield and imaging agent uptake. The fluorescence characteristic is preferably independent of the intensity of the emission and independent of imaging agent concentration.

The quantifying of step (v) preferably comprises: (a) establishing an estimate of the values, (b) determining a calculated emission as a function of the estimate, (c) comparing the calculated emission to the emission of said detecting to determine an error, (d) providing a modified estimate of the fluorescence characteristic as a function of the error. The quantifying preferably comprises determining the values from a mathematical relationship modelling multiple light-scattering behaviour of the tissue. The method of the first option preferably further comprises monitoring a metabolic property of the tissue in vivo by detecting variation of said fluorescence characteristic.

The optical imaging of the fifth aspect is preferably used to help facilitate the management of a disease state of the mammalian body. By the term “management” is meant use in the: detection, staging, diagnosis, monitoring of disease progression or the monitoring of treatment. The disease state is suitably one in which the enzyme which cleaves the second linkage of the probe is implicated. Imaging applications preferably include camera-based surface imaging, endoscopy and surgical guidance. Further details of suitable optical imaging methods have been reviewed by Sevick-Muraca et al [Curr. Opin. Chem. Biol., 6, 642-650 (2002)].

In a sixth aspect, the present invention provides a method of detection, staging, diagnosis, monitoring of disease progression or monitoring of treatment of a disease state of the mammalian body which comprises the in vivo optical imaging method of the sixth aspect.

The invention is illustrated by the non-limiting Examples detailed below. Example 1 provides a prophetic synthesis of a probe of the invention.

### EXAMPLE 1

### Synthesis of a Cathepsin B Probe (Prophetic Example)

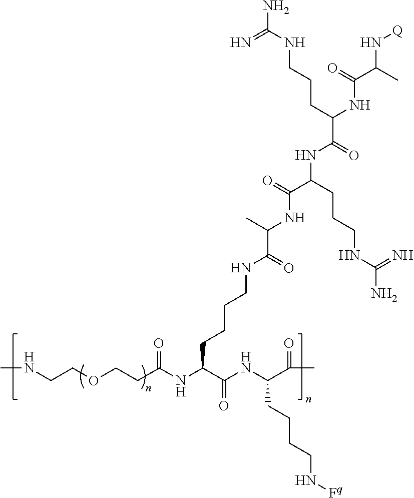 [0162] where:
Q is the quencher moiety;
F^{q} is the fluorochrome in a quenched relationship with Q;
n is an integer of value in the range 2 to 150.

The polymeric backbone is the polyethylene glycol (PEG) group. Attached thereto is the quencher moiety (Q) via a linkage which can be cleaved selectively by cathepsin B (Ala-Arg-Arg-Ala). Also attached to the PEG backbone is the fluorochrome (F^{q}). A specific compound is as follows:

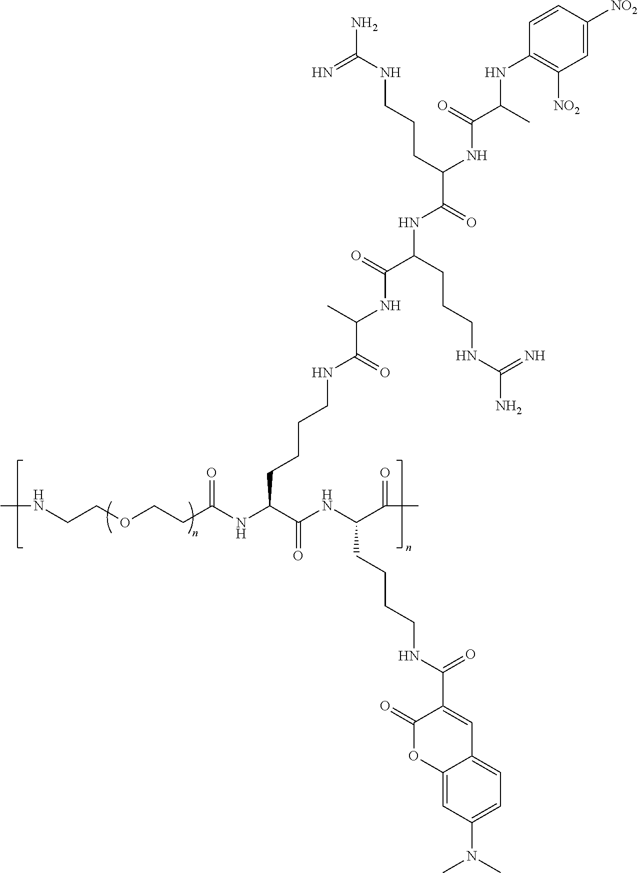

The quencher (Q) is a dinitrophenyl group, and the fluorochrome is a coumarin derivative.

What is claimed is:

1. An intramolecularly-quenched fluorescence probe comprising a polymeric backbone of molecular weight 10 to 100 kDa, where said backbone comprises a polypeptide copolymer, and:
(i) a number (z) of near-infrared fluorochromes each covalently linked to the backbone via a first linkage which is resistant to enzyme cleavage and biochemical oxidation;
(ii) a number (z) of quencher moieties each covalently linked to the backbone via a second linkage which is cleavable by either enzyme metabolism or biochemical oxidation;
wherein z is an integer of value 1 to 150, and wherein said quencher moieties are in a fluorescence-quenching energy transfer relationship with said fluorochromes.

2. The probe of claim 1, where the second linkage is cleavable by enzyme metabolism.

3. The probe of claim 2, where the enzyme is a hydrolytic enzyme.

4. The probe of claim 1 wherein the polymeric backbone is resistant to enzyme cleavage.

5-6. (canceled)

7. The probe of claim 1, wherein the copolymer comprises a lysine-glutamic acid copolymer.

8. The probe of claim 1, wherein the quencher moiety is chosen from:
(i) a non-fluorescent dye;
(ii) a nitro-substituted phenyl moiety;
(iii) an azulene dimer.

9. The probe of claim 1 wherein the quencher moiety is non-fluorescent.

10. The optical imaging agent of claim 1, wherein the quencher is biocompatible.

11. The probe of claim 1 wherein the first linkage comprises a sulfonamide or amide bond.

12. The probe of claim 1 wherein the fluorochrome is a fluorescent dye having an absorbance maximum in the range 600-1000 nm.

13. The probe of claim 1, further comprising a biological targeting molecule.

14. An optical imaging agent suitable for in vivo imaging which comprises the probe of claim 1.

15. The optical imaging agent of claim 14, where the probe is provided as a pharmaceutical composition which comprises the probe together with a biocompatible carrier, in a form suitable for mammalian administration.

16. (canceled)

17. A kit for the preparation of an optical imaging agent composition suitable for in vivo imaging, which comprises the probe of claim 1 in sterile, solid form such that upon reconstitution with a sterile supply of the biocompatible carrier, dissolution occurs to give the desired composition.

18. The kit of claim 17, where the sterile, solid form is a lyophilised solid.

19. An in vivo optical imaging method comprising:
(i) administering to a living animal or human subject an optical imaging agent suitable for in vivo imaging, which comprises the probe of claim 1;
(ii) allowing time for (a) the probe to accumulate in a target tissue of interest within said subject, and (b) enzymes in said target tissue to activate the probe by enzymatic cleavage at one or more of the second linkages as defined in claim 1, to give the activated probe;
(iii) illuminating the target tissue with a near-infrared excitation light of a wavelength absorbable by the fluorochrome of said activated probe;
(iv) fluorescence from the activated probe, which is generated by excitation of the fluorochrome in step (iii) is detected using a fluorescence detector;
(v) the light detected by the fluorescence detector is optionally filtered to separate out the fluorescence component; and
(vi) forming an optical image of the target tissue from the detected fluorescence of steps (iv) or (v).

20. The method of claim 19 where the excitation light of step (iii) is continuous wave (CW) in nature.

21. An in vivo optical imaging method comprising:
(i) administering to a living animal or human subject an optical imaging agent suitable for in vivo imaging, which comprises the probe of claim 1;
(ii) allowing time for (a) the probe to accumulate in a target tissue of interest within said subject, and (b) enzymes in said target tissue to activate the probe by enzymatic cleavage at one or more of the second linkages as defined in claim 1, to give the activated probe;
(iii) exposing light-scattering biologic tissue of said subject having a heterogeneous composition to excitation light from a light source with a pre-determined time varying intensity to excite the imaging agent, the tissue multiply-scattering the excitation light;
(iv) detecting a multiply-scattered light emission from the tissue in response to said exposing;
(v) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(vi) generating an image of the tissue by mapping the heterogeneous composition of the tissue in accordance with the values of step (v).

22. (canceled)

23. A method of detection, staging, diagnosis, monitoring of disease progression or monitoring of treatment of a disease state of the mammalian body which comprises the in vivo optical imaging method of claim 19.

24. A method of detection, staging, diagnosis, monitoring of disease progression or monitoring of treatment of a disease state of the mammalian body which comprises the in vivo optical imaging method of claim 21.


SEQUENCE LISTING (66 sequences)

SEQ ID NO: 1 - protein, 6 aa, Artificial
  synthetic peptide
PLXWAR

SEQ ID NO: 2 - protein, 7 aa, Artificial
  synthetic peptide
KPLXDAR

SEQ ID NO: 3 - protein, 5 aa, Artificial
  synthetic peptide
PLXAR

SEQ ID NO: 4 - protein, 9 aa, Artificial
  synthetic peptide
RPKPYXWMK

SEQ ID NO: 5 - protein, 9 aa, Artificial
  synthetic peptide
RPKPVXWRK

SEQ ID NO: 6 - protein, 8 aa, Artificial
  synthetic peptide
RPLXWRXC

SEQ ID NO: 7 - protein, 7 aa, Artificial
  synthetic peptide
RPLXWRS

SEQ ID NO: 8 - protein, 7 aa, Artificial
  synthetic peptide
PLGLXAR

SEQ ID NO: 9 - protein, 9 aa, Artificial
  synthetic peptide
SGKGPRXTA

SEQ ID NO: 10 - protein, 9 aa, Artificial
  synthetic peptide
SGKIPRXTA

SEQ ID NO: 11 - protein, 5 aa, Artificial
  synthetic peptide
PQGIA

SEQ ID NO: 12 - protein, 5 aa, Artificial
  synthetic peptide
PXXHA

SEQ ID NO: 13 - protein, 5 aa, Artificial
  synthetic peptide
PLXAR

SEQ ID NO: 14 - protein, 8 aa, Artificial
  synthetic peptide
GPLGMRGL

SEQ ID NO: 15 - protein, 7 aa, Artificial
  synthetic peptide
PGSGXAG

SEQ ID NO: 16 - protein, 13 aa, Artificial
  synthetic peptide
PGSGXASGTTGTG

SEQ ID NO: 17 - protein, 4 aa, Artificial
  synthetic peptide
SGXA

SEQ ID NO: 18 - protein, 4 aa, Artificial
  synthetic peptide
GSGX

SEQ ID NO: 19 - protein, 3 aa, Artificial
  synthetic peptide
FXR

SEQ ID NO: 20 - protein, 4 aa, Artificial
  synthetic peptide
XTPR

SEQ ID NO: 21 - protein, 4 aa, Artificial
  synthetic peptide
ARRA

SEQ ID NO: 22 - protein, 9 aa, Artificial
  synthetic peptide
GSPAFLAKR

SEQ ID NO: 23 - protein, 11 aa, Artificial
  synthetic peptide
GKPILFFRLKR

SEQ ID NO: 24 - protein, 8 aa, Artificial
  synthetic peptide
KPLGLXAR

SEQ ID NO: 25 - protein, 8 aa, Artificial
  synthetic peptide
KPLGLXAR

SEQ ID NO: 26 - protein, 7 aa, Artificial
  synthetic peptide
PXGXHAX

SEQ ID NO: 27 - protein, 12 aa, Artificial
  synthetic peptide
GGVVIATVKRRR

SEQ ID NO: 28 - protein, 7 aa, Artificial
  synthetic peptide
TVADAPK

SEQ ID NO: 29 - protein, 7 aa, Artificial
  synthetic peptide
DEVDAPK

SEQ ID NO: 30 - protein, 4 aa, Artificial
  synthetic peptide
DEVD

SEQ ID NO: 31 - protein, 9 aa, Artificial
  synthetic peptide
APAKFFRLK

SEQ ID NO: 32 - protein, 7 aa, Artificial
  synthetic peptide
PXGCHAK

SEQ ID NO: 33 - protein, 11 aa, Artificial
  synthetic peptide
LAQAVRSSSRX

SEQ ID NO: 34 - protein, 5 aa, Artificial
  synthetic peptide
GFSPX

SEQ ID NO: 35 - protein, 12 aa, Artificial
  synthetic pepide
AXGPQGFQGNXG

SEQ ID NO: 36 - protein, 9 aa, Artificial
  synthetic peptide
XAIKFFSRQ

SEQ ID NO: 37 - protein, 4 aa, Artificial
  synthetic peptide
RXXX

SEQ ID NO: 38 - protein, 4 aa, Artificial
  synthetic peptide
XRRX

SEQ ID NO: 39 - protein, 3 aa, Artificial
  synthetic peptide
XKX

SEQ ID NO: 40 - protein, 4 aa, Artificial
  synthetic peptide
PFRX

SEQ ID NO: 41 - protein, 4 aa, Artificial
  synthetic peptide
VLRX

SEQ ID NO: 42 - protein, 5 aa, Artificial
  synthetic peptide
RGFFX

SEQ ID NO: 43 - protein, 3 aa, Artificial
  synthetic peptide
AAF

SEQ ID NO: 44 - protein, 4 aa, Artificial
  synthetic peptide
AGLA

SEQ ID NO: 45 - protein, 11 aa, Artificial
  synthetic peptide
PKPQQFFGLXG

SEQ ID NO: 46 - protein, 10 aa, Artificial
  synthetic peptide
RPKPVDXWRX

SEQ ID NO: 47 - protein, 11 aa, Artificial
  synthetic peptide
XGPDGFQGNXG

SEQ ID NO: 48 - protein, 9 aa, Artificial
  synthetic peptide
LRLSSYYSG

SEQ ID NO: 49 - protein, 4 aa, Artificial
  synthetic peptide
FRRX

SEQ ID NO: 50 - protein, 4 aa, Artificial
  synthetic peptide
GPRX

SEQ ID NO: 51 - protein, 4 aa, Artificial
  synthetic peptide
RRXR

SEQ ID NO: 52 - protein, 14 aa, Artificial
  synthetic peptide
GISSFYSSTEERLW

SEQ ID NO: 53 - protein, 20 aa, Artificial
  synthetic peptide
PLPRNXTEXEARGXVILTXK

SEQ ID NO: 54 - protein, 5 aa, Artificial
  synthetic peptide
YIGSR

SEQ ID NO: 55 - protein, 5 aa, Artificial
  synthetic peptide
PDSGR

SEQ ID NO: 56 - protein, 5 aa, Artificial
  synthetic peptide
IKVAV

SEQ ID NO: 57 - protein, 3 aa, Artificial
  synthetic peptide
LRE

SEQ ID NO: 58 - protein, 15 aa, Artificial
  synthetic peptide
KCQAGTFALRGDPQG

SEQ ID NO: 59 - protein, 3 aa, Artificial
  synthetic peptide
RGD

SEQ ID NO: 60 - protein, 8 aa, Artificial
  synthetic peptide
DRVYIHPF

SEQ ID NO: 61 - protein, 10 aa, Artificial
  synthetic peptide
XARGVYIHPI

SEQ ID NO: 62 - protein, 10 aa, Artificial
  synthetic peptide
DRVYIHPFHL

SEQ ID NO: 63 - protein, 21 aa, Artificial
  synthetic peptide
CSCSSLMDKECVYFCHLDIIW

SEQ ID NO: 64 - protein, 14 aa, Artificial
  synthetic peptide
XQRLGNQWAVGHLM

SEQ ID NO: 65 - protein, 16 aa, Artificial
  synthetic peptide
GPWLEEEEEAYGWMDF

SEQ ID NO: 66 - protein, 9 aa, Artificial
  synthetic peptide
HWSYGLRPG